(19) United States
(12) Patent Application Publication
(10) Pub. No.: US 2025/0327814 A1
(43) Pub. Date: Oct. 23, 2025

(54) METHOD FOR TESTING TERTIARY LYMPHOID TISSUE, AND KIT FOR TESTING TERTIARY LYMPHOID TISSUE

(71) Applicant: KYOTO UNIVERSITY, Kyoto-shi, Kyoto (JP)

(72) Inventors: Motoko YANAGITA, Kyoto-shi (JP); Yuki SATO, Kyoto-shi (JP); Makiko KONDO, Kyoto-shi (JP); Ryo KAMIMATSUSE, Kyoto-shi (JP)

(73) Assignee: KYOTO UNIVERSITY, Kyoto-shi, Kyoto (JP)

(21) Appl. No.: 18/870,759

(22) PCT Filed: Jun. 5, 2023
(86) PCT No.: PCT/JP2023/020894

§ 371 Date: Dec. 2, 2024

(30) Foreign Application Priority Data
Jun. 3, 2022 (JP) 2022-091000

(51) Int. Cl. G01N 33/68; G01N 33/70

(52) U.S. Cl. CPC G01N 33/6863; G01N 33/70; G01N 2333/70578; G01N 2800/347

(57) ABSTRACT

One purpose of the present disclosure is to provide a technology for testing the presence or absence of a TLT in a subject without biopsy. Disclosed is a method for testing the presence or absence of a tertiary lymphoid tissue in a subject, the method including the step of measuring a soluble CD30 concentration in a blood sample collected from the subject.

### TECHNICAL FIELD

The present disclosure relates to a method for testing the presence or absence of a tertiary lymphoid tissue (TLT) in a subject. In addition, the present disclosure also relates to a kit for testing the presence or absence of a TLT in a subject. Further, the present disclosure relates to a test method for predicting the prognosis of renal function in a subject. Furthermore, the present disclosure relates to a test kit for predicting the prognosis of renal function in a subject.

### BACKGROUND ART

TLT is an inducible ectopic lymphoid tissue in which a tissue similar to a lymph node is formed in a non-lymphoid tissue (Non-Patent Literatures 1 to 3). TLT has a large number of lymphocytes infiltrated, and acts as an origin of acquired immune response via interaction and proliferation of T cells and B cells to amplify an immune response. It is known that TLT is formed in a lesion of, for instance, a disease accompanied by chronic inflammation, an autoimmune disease, an infectious disease, or cancer (Non-Patent Literatures 1 to 3), and TLT is found to affect the severity and prognosis of these diseases.

For example, in kidney diseases such as pyelonephritis. IgA nephropathy, and lupus nephritis, it is known that protraction and repair failure of inflammation occur when a TLT is induced (Non-Patent Literature 4). In addition, in recent years, the present inventors have revealed that the stage of TLT maturation can be used as a biomarker reflecting the degree of tissue injury of the kidney (Non-Patent Literature 5). Specifically, the results of tissue analysis of human pyelonephritis have indicated a simultaneous and frequent occurrence of TLTs at various maturation stages in the kidney. A more mature TLT is observed in a lesion where tissue damage is severe. On the other hand, it has been revealed that the induction of TLT is inconspicuous in a site where the injury is mild, and the proportion of immature TLTs is also high in the formed TLTs. In addition, even in the analysis of the elderly, it has been found that the number of TLTs is significantly larger in the cases of complicated chronic kidney disease than in the non-complicated cases, and the proportion of more mature TLTs is high. Further, it has also been found that TLTs are induced to the same anatomical site through the same maturation stages, regardless of the type of underlying disease. A report (Non-Patent Literature 6) shows that when mature TLTs are present in the kidney after transplantation, deterioration of renal function after 5 years is likely to be observed. Thus, TLT can be considered to be a predictive marker of future renal function.

Here, TLT has been known to be able to affect the severity, the prognosis, and the treatment responsiveness of the diseases. It is useful to check the presence or absence of a TLT formed and the stage of maturation for determining a strategy regarding therapeutic intervention and judging the effect.

Meanwhile, in order to check the presence or absence of a TLT formed and the stage of maturation, it is necessary to collect a lesion tissue and perform biopsy. However, during the biopsy of the lesion, it is necessary to keep the posture constant for a predetermined time, a risk of, for instance, bleeding is present, and there is a disadvantage that the burden on the patient is large. Besides, in patients being treated with an antiplatelet drug or the elderly, a biopsy risk is high. In these patients, it is particularly difficult to check the presence or absence of a TLT formed and the stage of maturation.

### CITATION LIST

### Non Patent Literature

Non-Patent Literature 1: Sato Y. et al., Immunology of the ageing kidney. Nat Rev Nephrol. 2019; 15(10): 625-40.
Non-Patent Literature 2: Antonioli L. et al. Ectopic Lymphoid Organs and Immune-Mediated Diseases: Molecular Basis for Pharmacological Approaches. Trends Mol Med. 2020; 26(11): 1021-1033.
Non-Patent Literature 3: Sautes-Fridman C. et al. Tertiary lymphoid structures in theera of cancer immunotherapy. Nat Rev Cancer. 2019; 19(6): 307-25.
Non-Patent Literature 4: Sato Y. et al. Heterogeneous fibroblasts underlie age-dependent tertiary lymphoid tissues in the kidney. JCI Insight. 2016; 1(11): e87680.
Non-Patent Literature 5: Sato Y. et al. Developmental stages of tertiary lymphoid tissue reflect local injury and inflammation in mouse and human kidneys. Kidney Int. 2020; 98(2): 448-463.
Non-Patent Literature 6: Lee H. et al., Advanced Tertiary Lymphoid Tissues in Protocol Biopsies are Associated with Progressive Graft Dysfunction in Kidney Transplant Recipients. J Am Soc Nephrol. 2022; 33(1): 186-200. doi: 10.1681/ASN.2021050715.

### SUMMARY OF INVENTION

### Technical Problem

One purpose of the present disclosure is to provide a technology for testing the presence or absence of a TLT in a subject without biopsy. Another purpose of the present disclosure is to provide a test technology for predicting the prognosis of renal function in a subject.

### Solution to Problem

The present inventors have conducted intensive research to solve the above problems, and have found that: the soluble CD30 (sCD30) concentration in a blood sample collected from a subject correlates with the presence or absence and the stage of maturation of a TLT; the sCD30 concentration is higher in those with a TLT(s) formed than in those without any TLT formed; and the higher the stage of TLT maturation, the higher the sCD30 concentration. It has thus been discovered that the sCD30 concentration in a blood sample can be used as a biomarker for testing the presence or absence and the stage of maturation of a TLT.

Further, the present inventors have found that the estimated glomerular filtration rate (eGFR) and the creatinine concentration in a blood sample of the subject correlate with the presence or absence of a TLT in the kidney; and those with a TLT(s) formed have a lower eGFR and a higher creatinine concentration in a blood sample than those without any TLT formed. It has thus been discovered that the eGFR or the creatinine concentration in a blood sample can be used as a biomarker for testing the presence or absence of a TLT.

Furthermore, the present inventors have found that an event in which eGFR decreases during a prognosis is likely to occur in IgA nephropathy patients having a high sCD30 concentration in a blood sample. It has thus been discovered that the sCD30 concentration in a blood sample can be used as a biomarker for predicting the prognosis of renal function.

The present disclosure has been completed by conducting further studies based on the findings. Specifically, the present disclosure provides the following items of the invention.

Item 1. A test method for testing a presence or absence of a tertiary lymphoid tissue in a subject, including the step of measuring a soluble CD30 concentration in a blood sample collected from the subject.

Item 2. The test method according to item 1, wherein the subject is a kidney disease patient, a person who needs to be tested for a presence or absence of kidney disease, or a person who has received a kidney transplant, and the presence or absence of a tertiary lymphoid tissue in a kidney is tested.

Item 3. The test method according to item 1 or 2, wherein the blood sample is serum.

Item 4. The test method according to any one of items 1 to 3, further including a step of measuring a creatinine concentration in serum collected from the subject to calculate an estimated glomerular filtration rate of the subject.

Item 5. A test kit for testing a presence or absence of a tertiary lymphoid tissue in a body, including a reagent for measuring a soluble CD30 concentration.

Item 6. The kit according to item 5, further including a reagent for measuring a creatinine concentration.

Item 7. A method for testing a presence or absence of a tertiary lymphoid tissue in a kidney of a subject, including the step of measuring a creatinine concentration in serum collected from the subject to calculate an estimated glomerular filtration rate.

Item 8. A method for testing a presence or absence of a tertiary lymphoid tissue in a kidney of a subject, including the step of measuring a creatinine concentration in serum collected from the subject.

Item 9. A test kit for testing a presence or absence of a tertiary lymphoid tissue in a kidney, including a reagent for measuring a creatinine concentration.

Item 10. A test method for predicting a prognosis of renal function in a subject, including the step of measuring a soluble CD30 concentration in a blood sample collected from the subject.

Item 11. The test method according to item 10, wherein the subject is a kidney disease patient or a person who has received a kidney transplant.

Item 12. A test kit for predicting a prognosis of renal function in a subject, including a reagent for measuring a soluble CD30 concentration.

### Advantageous Effects of Invention

A test method according to an embodiment of the present disclosure enables the presence or absence of a TLT in a subject to be tested, without biopsy, by using the sCD30 concentration in a blood sample as a TLT biomarker. In addition, the sCD30 concentration in a blood sample also correlates with the stage of TLT maturation. Therefore, in the test method for the present disclosure, the sCD30 concentration in a blood sample can be used as a TLT biomarker. In this case, the stage of maturation of a TLT formed in a body can be predicted. For example, the test method of the present disclosure can be used to predict the presence or absence or the stage of maturation of a TLT in the kidney of a kidney disease patient or a person who has received a kidney transplant. This makes it possible to estimate, for instance, the degree of renal injury and/or deterioration in the function of the transplanted kidney.

In addition, a test method according to another embodiment of the present disclosure enables the presence or absence of TLT in a kidney to be tested, without biopsy, by using the eGFR or the creatinine concentration in a blood sample as a TLT biomarker.

Further, a test method according to still another embodiment of the present disclosure can predict the prognosis of the renal function in a kidney disease patient or a person who has received a kidney transplant by using the sCD30 concentration in a blood sample as a biomarker.

### BRIEF DESCRIPTION OF DRAWINGS

FIG. 1 is a schematic diagram of the structure of a TLT at each maturation stage as formed in the kidney.

FIG. 2 is a diagram showing a protocol in which 105 cases of IgA nephropathy are divided into a “TLT group” (46 cases) and a “non-TLT+small TLT group” (59 cases).

FIG. 3 is a graph showing the results of comparing the serum sCD30 concentration between the TLT group and the non-TLT+small TLT group.

FIG. 4 is a graph showing the results of comparing the serum sCD30 concentration among stage I at the TLT group, stage II at the TLT group, and the non-TLT+small TLT group.

FIG. 5 is graphs showing correlations between the serum sCD30 concentration, the serum creatinine (sCre) concentration, the urine protein/creatinine ratio (UPCR), and the age in 105 cases of IgA nephropathy.

FIG. 6 is a graph and a table showing the results of univariate analysis by logistic regression while using the serum sCD30 concentration as an explanatory variable, in which analysis patients in the TLT group are set as positive (1) and patients in the non-TLT+small TLT group are set as negative (0) in 105 cases of IgA nephropathy.

FIG. 7 The left graph of FIG. 7 is a graph showing the results of univariate analysis by logistic regression while using the eGFR as an explanatory variable, in which analysis patients in the TLT group are set as positive (1) and patients in the non-TLT+small TLT group are set as negative (0) in 105 cases of IgA nephropathy. The right graph is a graph showing the results of predicting the presence or absence of a TLT from multivariate analysis by logistic regression using eGFR and the serum sCD30 concentration after binarized (<36.719 ng/mL: 0; ≥36.719 ng/mL: 1) for 105 cases of IgA nephropathy.

FIG. 8 is graphs showing the results of predicting the presence or absence of a TLT from multivariate analysis by logistic regression using eGFR, age, UPCR, and the serum sCD30 concentration after binarized (<36.719 ng/mL: 0: ≥36.719 ng/mL: 1) for 105 cases of IgA nephropathy.

FIG. 9 is a graph showing the results of tracking the presence or absence of incidence of an event in which eGFR decreases by 20% or more over 5 years while dividing 105 cases of IgA nephropathy into the sCD30 high level group (serum sCD30 concentration is 36.719 ng/mL or more) and the sCD30 low level group (serum sCD30 concentration is less than 36.719 ng/mL). The vertical axis of FIG. 9 is the “Cumulative incidence of events with a decrease in eGFR by 20% or more”, which is calculated in each year by subtracting, from 1, a value obtained by multiplying the event-free rate up to the previous year by the event-free rate in the corresponding year. The event-free rate is the ratio of the number of patients who did not experience an event with a decrease in eGFR by 20% or more in the corresponding year to the number of patients who were able to be tracked in that year (patients whose eGFR decreased by 20% or more by the previous year and patients whose tracking were terminated immediately before that year are excluded). In addition, in FIG. 9, the numerical value indicated in the column of “No. at risk” is the number of patients at risk immediately before each time point, that is, the number of patients who were able to be tracked immediately before that time point without occurrence of an event with a decrease in eGFR by 20% or more.

### DESCRIPTION OF EMBODIMENTS

### 1. Terms

Unless specifically defined otherwise, the terms used herein have the meanings as commonly understood by those skilled in the art of medicine, pharmacy, molecular biology, microbiology, organic chemistry, and so on. When a term defined herein does not have the same meaning as commonly understood, the description herein prevails.

In the present disclosure, a “tertiary lymphoid tissue (TLT)” is a tissue similar to a lymph node, and is an inducible ectopic lymphoid tissue composed of an aggregate of lymphocytes and formed in a non-lymphoid tissue.

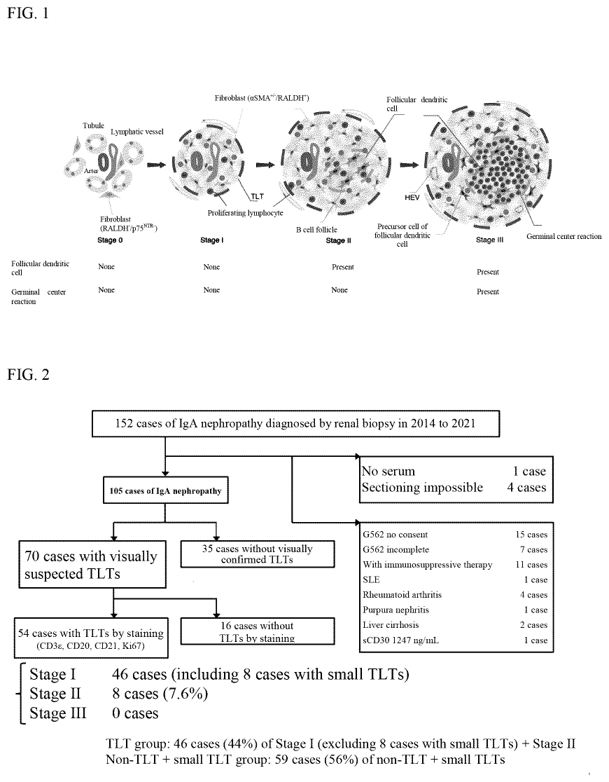

In the present disclosure, the “stage of maturation of a tertiary lymphoid tissue (TLT)” refers to the degree of structural maturation of a TLT. In the present disclosure, the stage of maturation of a tertiary lymphoid tissue (TLT) is classified into stages I to III below according to the classification described in Non-Patent Literature 5. For reference, FIG. 1 shows a schematic diagram of the structure of a TLT at each maturation stage as formed in the kidney. Stage 0 shown in FIG. 1 is a TLT precursor in which lymphocytes are not organized, and does not correspond to a TLT in the present disclosure.
Stage I: TLT without follicular dendritic cells (FDCs) or a germinal center
Stage II: TLT with FDCs but without a germinal center
Stage III: TLT containing FDCs and a germinal center

In the present disclosure, the “soluble CD30 (sCD30)” is a soluble molecule composed of the extracellular domain of CD30.

In the present disclosure, the “estimated glomerular filtration rate (eGFR)” is a glomerular filtration rate (unit: mL/min/1.73 m²) calculated on the basis of a serum creatinine concentration in consideration of age and sex. The value is calculated according to the calculation formula below as corrected such that the test subjects aged 18 or more are directed to Japanese people proposed by the Japanese Society of Nephrology. The value for subjects aged less than 18 is calculated according to the calculation formula below as proposed by the Japanese Society for Pediatric Nephrology.

$$[ Mathematical Formula 1 ]$$

$$In the cases of male subjects aged 18 or more$$

$$eGFR ( mL / min / 1.73 {m}^{2} ) = 194 \times {sCr}^{- 1.094} \times {age}^{- 0.286}$$

$$In the cases of female subjects aged 18 or more$$

$$eGFR ( mL / min / 1.73 {m}^{2} ) = 194 \times {sCr}^{- 1.094} \times {age}^{- 0.286} \times 0739$$

$$In the cases of young males aged less than 18$$

$$eGFR ( mL / min / 1.73 {m}^{2} ) = 110.2 \times ( refCr / sCr ) + 2.93$$

$$refCr = - 1.259 {Ht}^{5} + 7.815 {Ht}^{4} - 18.57 {Ht}^{3} + 21.39 {Ht}^{2} - 11.71 Ht + 2.628$$

$$In the cases of young females aged less than 18$$

$$eGFR ( mL / min / 1.73 {m}^{2} ) = 110.2 \times ( refCr / sCr ) + 2.93$$

$$refCr = - 4.536 {Ht}^{5} + 27.16 {Ht}^{4} - 63.47 {Ht}^{3} + 72.43 {Ht}^{2} - 40.06 Ht + 8.778$$

$$sCr : Serum creatinine concentration ( mg / dL )$$

$$refCr : Serum creatinine concentration reference value ( mg / dL ) calculated from height$$

$$Ht : Height ( m )$$

In the present disclosure, the “creatinine” is a metabolite of creatine phosphate, and is a component observed in blood and urine.

### 2. TLT Test Technology Using sCD30 Concentration in Blood Sample as Biomarker

### 2-1. Test Method 1

An embodiment of the present disclosure provides a method for testing the presence or absence of a TLT in a subject (hereinafter, sometimes referred to as “test method 1”), the method including a step of measuring a sCD30 concentration in a blood sample collected from the subject.

### [Subject]

In test method 1 of the present disclosure, the “subject” is a human or non-human animal to be tested for the presence or absence of a TLT formed in a body. Examples of the non-human animal include non-human mammals such as primates, rats, mice, gerbils, guinea pigs, hamsters, ferrets, rabbits, cows, horses, pigs, goats, dogs, and cats. Suitable examples of the subject include a human.

The subject in the test method 1 of the present disclosure may be any subject as long as the subject needs to be tested for the presence or absence of a TLT formed in a body. However, suitable examples include a person suffering from a disease in which a TLT is formed at a lesion, a person who needs to be tested for the presence or absence of the disease, or a person who has received an organ transplant for the treatment of the disease. Examples of the disease in which a TLT is formed at a lesion include a disease accompanied by chronic inflammation, chronic kidney disease, obesity, asthma, allergic lung disease, arteriosclerosis, autoimmune disease, infectious disease, or cancer.

Specific examples of the subject in test method 1 of the present disclosure include a kidney disease patient or a person who needs to be tested for the presence or absence of kidney disease. The type of kidney disease is not particularly limited, and examples thereof include IgA nephropathy, lupus nephritis, ANCA-associated vasculitis, diabetic nephropathy, nephrosclerosis, pyelonephritis, or chronic renal failure. Among these kidney diseases. IgA nephropathy is preferable. In kidney diseases, as renal dysfunction progresses, a TLT is likely to be formed in a lesion, and its stage of maturation tends to increase. Therefore, in test method 1 of the present disclosure, when the subject is a person suffering from kidney disease or a person who needs to be tested for the presence or absence of kidney disease, the presence or absence of kidney disease and the degree of renal dysfunction can be estimated in addition to the presence or absence of a TLT.

Specific examples of the subject in test method 1 of the present disclosure include a person whose future renal function needs to be predicted. Examples of the person whose future renal function needs to be predicted include a kidney disease patient or a person who has received a kidney transplant. Even if the renal function is normal at the time of testing, a TLT(s) may be formed in the kidney. As the stage of maturation becomes higher, the renal function tends to deteriorate more in the future. Test method 1 of the present disclosure can thus be used for prediction of the future renal function. For example, in a case where the subject is a person who has received a kidney transplant, the prognosis of the function of the transplanted kidney can be predicted according to the presence or absence of a TLT and the stage of maturation thereof.

Specific examples of the subject in test method 1 of the present disclosure include a hepatitis C patient or a person who needs to be tested for the presence or absence of hepatitis C. As the symptoms of hepatitis C progress, a TLT is more likely to be formed in the liver, and its stage of maturation tends to increase. Therefore, in test method 1 of the present disclosure, when the subject is a person suffering from hepatitis C or a person who needs to be tested for the presence or absence of hepatitis C, the presence or absence of hepatitis C and the degree of hepatitis C symptoms can be estimated in addition to the presence or absence of a TLT.

Specific examples of the subject in test method 1 of the present disclosure include a cancer patient. The type of cancer is not particularly limited, and examples thereof include solid cancers such as kidney cancer, gastric cancer, lung cancer, breast cancer, liver cancer, tongue cancer, thyroid cancer, uterine cancer, ovarian cancer, or prostate cancer. When a TLT(s) is formed in a cancer tissue and the stage of maturation thereof increases, the cancer immune response is enhanced, and there is a tendency that the therapeutic effect of cancer is improved and the prognosis is found to be good. Therefore, in test method 1 of the present disclosure, when the subject is a cancer patient, particularly a cancer patient who receives an immune checkpoint inhibitor, the therapeutic effect and prognosis of cancer can be predicted.

### [Blood Sample]

In the present disclosure, the “blood sample” is a sample derived from blood, and examples thereof include whole blood, serum, or plasma. The blood sample used in test method 1 of the present disclosure may be either whole blood, serum, or plasma, but serum is a suitable example.

[To Measure sCD30]

In test method 1 of the present disclosure, in order to use the sCD30 concentration in a blood sample as a TLT biomarker, the sCD30 concentration in a blood sample is measured. The sCD30 concentration in a blood sample can be measured by, for example, an immunoassay using an antibody that specifically recognizes and binds to sCD30. The antibody may be produced by a known procedure. Examples of the immunoassay include an assay using a solid phase carrier on which an antibody specifically binding to sCD30 is immobilized, flow cytometry, or Western blotting. Examples of the assay using a solid phase carrier include an enzyme immunoassay (ELISA) using an immobilized microtiter plate, or a precipitation method (immunoprecipitation method) using immobilized particles. The sCD30 concentration in a blood sample can also be measured by a method using, for instance, multiple reaction monitoring (MRM) by liquid chromatograph mass spectrometry (LC-MS/MS), which is a protein mass spectrometry technique without using an antibody. These detection methods can also be performed by conventional procedures.

In addition, since a kit for measuring sCD30 is commercially available, the sCD30 concentration in a blood sample can be conveniently measured using the commercially available kit.

### [To Determine, for Instance, Presence or Absence of TLT]

The presence or absence of a TLT is correlated with the sCD30 concentration in a blood sample. When any TLT is not formed in a body, the sCD30 concentration in a blood sample is low, and when a TLT(s) is formed in a body, the sCD30 concentration in a blood sample is high. Therefore, in test method 1 of the present disclosure, it is determined that the lower the sCD30 concentration in a blood sample, the higher the possibility that any TLT is not formed in a body, and the higher the sCD30 concentration in a blood sample, the higher the possibility that a TLT(s) is formed in a body.

The sCD30 concentration in a blood sample is highly correlated with the presence or absence of a TLT composed of an aggregate of more than 120 lymphocytes. Therefore, test method 1 according to an embodiment of the present disclosure is performed in order to test the presence or absence of a TLT composed of an aggregate of more than 120 lymphocytes.

Test method 1 of the present disclosure may be used to determine the presence or absence of a TLT on the basis of the sCD30 concentration in a blood sample by comparing it with a reference value obtained in advance from those in which the presence or absence of a TLT has been found. Here, the “reference value” is a value serving as a reference for determining the presence or absence of a TLT. Specifically, it is, for instance, the median of the sCD30 concentration in blood samples obtained from those in which the presence or absence of a TLT has been found or a cut-off value obtained from the sCD30 concentrations in said blood samples.

For example, the median of the sCD30 concentration in blood samples derived from those without a TLT formed in a body is obtained in advance, and this median is then used as a reference value. When the sCD30 concentration in a blood sample of a subject is equal to or less than the reference value, it can be determined that the subject might not have any TLT formed in a body. In addition, for example, the median of the sCD30 concentration in blood samples derived from those with a TLT formed in a body is obtained in advance, and this median is then used as a reference value. When the sCD30 concentration in a blood sample of a subject is equal to or more than the reference value, it can be determined that the subject might have a TLT(s) formed in a body. In addition, for example, the median of the sCD30 concentration in blood samples derived from those without a TLT formed in a body and the median of the sCD30 concentration in blood samples derived from those with a TLT(s) formed in a body are obtained in advance, and then a cut-off value for distinguishing the presence or absence of a TLT is determined from the sCD30 concentrations, and the cut-off value is then used as a reference value. When the sCD30 concentration in a blood sample of a subject is equal to or more than the cut-off value, it can be determined that the subject might have a TLT(s) formed in a body.

The reference value is desirably obtained for each type of disease to be tested. For example, when the subject is a kidney disease patient or a person who needs to be tested for the presence or absence of kidney disease, the reference values used may be the median of the sCD30 concentration in blood samples from kidney disease patients in which any TLT is not found to be formed in the kidney, the median of the sCD30 concentration in blood samples from kidney disease patients in which a TLT(s) is found to be formed in the kidney, and/or the cut-off value determined from these sCD30 concentrations. For example, when the subject is a hepatitis C patient or a person who needs to be tested for the presence or absence of hepatitis C, the reference values used may be the median of the sCD30 concentration in blood samples from hepatitis C patients in which any TLT is not found to be formed in the liver, the median of the sCD30 concentration in blood samples from hepatitis C patients in which a TLT(s) is found to be formed in the liver, and/or the cut-off value determined from these sCD30 concentrations.

Further, as shown in the section of Examples, 105 cases of kidney disease patients were analyzed by the present inventors. As a result, the median of the serum sCD30 concentration of a kidney disease patient group in which a TLT (TLT composed of an aggregate of more than 120 lymphocytes) was formed in the kidney was 39.8 ng/mL; the median of the serum sCD30 concentration of a kidney disease patient group in which no TLT was formed in the kidney or only an immature small TLT (TLT composed of an aggregate of 120 lymphocytes or less) was formed in the kidney was 31.7 ng/mL; and the cut-off value was determined to be 36.719 ng/mL at which the two groups were able to be distinguished with a sensitivity of 60% and a specificity of 76%. Therefore, when test method 1 of the present disclosure is applied to the test for the presence or absence of a TLT in the kidney, these serum sCD30 concentrations or the cut-off value can also be used as reference values.

Furthermore, the sCD30 concentrations in blood samples from those in which any TLT is not formed in a body or in blood samples from those in which a TLT(s) is formed in a body are obtained in advance. The presence or absence of a TLT may be predicted from the sCD30 concentration in a blood sample of a subject by using the analysis results of logistic regression analysis using these data.

The stage of TLT maturation is correlated with the sCD30 concentration in a blood sample. The higher the stage of maturation of the TLT formed in a body, the higher the sCD30 concentration in a blood sample. Therefore, the stage of maturation of the TLT formed in a body can also be determined by test method 1 of the present disclosure.

In order to determine the stage of maturation of the TLT formed in a body by test method 1 of the present disclosure, the stage can be determined by comparison with a reference value obtained in advance from those in which a TLT(s) is formed in a body and the stage of maturation is known. Here, the “reference value” is a value serving as a reference for determining the stage of maturation of the TLT, and specifically, is the median of the sCD30 concentration in blood samples obtained from those in which a TLT(s) is formed in a body and the stage of maturation is known or a cut-off value obtained from the sCD30 concentrations in the blood samples and so on.

For example, blood samples derived from those in which a TLT(s) is formed in a body may be used to determine, in advance, the median of the sCD30 concentration for a TLT at each maturation stage or a cut-off value of the sCD30 concentration for a TLT at each maturation stage. This may be used as a reference value to determine which maturation stage the sCD30 concentration in a blood sample of a subject corresponds to. In addition, the maturation stage of a TLT may be predicted from the sCD30 concentration in a blood sample of a subject by using the analysis results of the logistic regression analysis.

As shown in the section of Examples, 46 kidney disease patients in which a TLT (TLT composed of an aggregate of more than 120 lymphocytes) was formed in the kidney were analyzed by the present inventors. As a result, the median of the serum sCD30 concentration of a kidney disease patient group in which a TLT at stage I was formed was 38.8 ng/mL; and the median of the serum sCD30 concentration of a kidney disease patient group in which a TLT at stage II was formed was found to be 50.5 ng/mL. Therefore, these serum sCD30 concentrations may be used as reference values to predict the stage of maturation of the TLT formed in the kidney.

Based on the presence or absence of a TLT in a body or the stage of maturation of a TLT as predicted by test method 1 of the present disclosure, it is possible to estimate the presence or absence of a disease in which a TLT is formed at a lesion or the degree of symptom of the disease.

For example, in test method 1 of the present disclosure, when a subject suffers from a non-cancer disease with a TLT formed and may have a possibility of having a TLT(s) formed in a body, it is presumed that the disease has progressed, and it is predicted that the higher the stage of maturation of the TLT, the higher the degree of progression of the disease. Specifically, in test method 1 of the present disclosure, when the subject is a kidney disease patient and may be predicted to be at risk of having a TLT(s) formed in a body, it is presumed that the kidney disease has progressed, and it is also presumed that the higher the stage of maturation of the TLT is, the severer the renal dysfunction is.

In addition, a therapeutic agent targeting a TLT may be effective for a disease accompanied by formation of a TLT (other than a disease accompanied by cancer and/or infection), and development of the therapeutic agent targeting a TLT is expected in the future. Therefore, test method 1 of the present disclosure can also be used for determining whether to administer a TLT-targeting therapeutic agent that can be developed in the future. Specifically, in test method 1 of the present disclosure, when the subject suffers from a disease accompanied by formation of a TLT, which disease is other than a disease accompanied by cancer and/or infection, and may be predicted to have a possibility of having a TLT(s) formed in a body, it can be determined that the patient is suitable for administration of a therapeutic agent targeting a TLT. Note that in the present disclosure, the “therapeutic agent targeting a TLT” is a drug that stops or suppresses the progression of a TLT or causes the disappearance or amelioration of a TLT.

Further, in test method 1 of the present disclosure, when the subject is a person, whose future renal function needs to be predicted and whose renal function is normal at the time of the test, and may be predicted to be at risk of having a TLT formed, it is presumed that there is a possibility of causing deterioration in renal function. In such case, it is presumed that the higher the stage of maturation of the TLT, the higher the possibility of causing deterioration in renal function. For example, when the subject is a person received a kidney transplant, whose renal function is normal at the time of the test, and may be predicted to be at risk of having a TLT formed, it is presumed that the prognosis of the function of the transplanted kidney may be worsened. In such case, it is presumed that the higher the stage of maturation of the TLT, the worse the prognosis of the function of the transplanted kidney may be.

Furthermore, in test method 1 of the present disclosure, when the subject is a cancer patient and may be predicted to have a possibility of having a TLT(s) formed in a body, it is presumed that the cancer immune response is enhanced. In such case, it is also presumed that the higher the stage of maturation of the TLT, the higher the therapeutic effect for cancer, and the better the prognosis.

### [Another Biomarker Used in Combination]

The present inventors have found that the presence or absence of a TLT in the kidney is correlated with eGFR; when any TLT is not formed in the kidney, eGFR is high; and when a TLT(s) is formed in the kidney, eGFR is low. Therefore, in test method 1 of the present disclosure, the eGFR together with the sCD30 concentration in a blood sample may be used as a TLT biomarker to increase the accuracy of predicting the presence or absence of a TLT in the kidney.

In test method 1 of the present disclosure, the presence or absence of a TLT in the kidney may be tested based on the eGFR by comparing it with a reference value obtained in advance from those in which the presence or absence of a TLT has been found. Here, the “reference value” is a value serving as a reference for determining the presence or absence of a TLT, and specifically, it is the median of eGFR obtained from those in which the presence or absence of a TLT has been found or a cut-off value calculated from the eGFR.

For example, the median of the eGFR of those without a TLT formed in the kidney is obtained in advance, and this median is then used as a reference value. When the eGFR of a subject is equal to or more than the reference value, it can be determined that the subject might not have any TLT formed in the kidney. For example, the median of the eGFR of those with a TLT formed in the kidney is obtained in advance, and this median is then used as a reference value. When the eGFR of a subject is equal to or less than the reference value, it can be determined that the subject might have a TLT(s) formed in the kidney. Further, for example, the eGFR of those without a TLT formed in the kidney and the eGFR of those with a TLT formed in the kidney are obtained in advance. A cut-off value for distinguishing the presence or absence of a TLT in the kidney is determined from these eGFR values, and the cut-off value is then used as a reference value. When the eGFR of a subject is equal to or less than the cut-off value, it can be determined that the subject might have a TLT(s) formed in the kidney. Furthermore, the eGFR of those without a TLT formed in the kidney and the eGFR of those with a TLT formed in the kidney are obtained in advance. The presence or absence of a TLT may be predicted from the eGFR of a subject by using the analysis results of logistic regression analysis using these data.

Further, as shown in the section of Examples, 105 cases of kidney disease patients were analyzed by the present inventors. As a result, the median of the eGFR of a kidney disease patient group in which a TLT (TLT composed of an aggregate of more than 120 lymphocytes) was formed in the kidney was 53.3 mL/min/1.73 m²; the median of the eGFR of a kidney disease patient group in which no TLT was formed in the kidney or only an immature small TLT (TLT composed of an aggregate of 120 lymphocytes or less) was formed in the kidney was 79.2 mL/min/1.73 m²; and the cut-off value was determined to be 59.3 mL/min/1.73 m² at which the two groups were able to be distinguished with a sensitivity of 65% and a specificity of 81%. Therefore, in test method 1 of the present disclosure, when eGFR is also used as a TLT biomarker, these eGFR values or the cut-off value can also be used as reference values.

In test method 1 of the present disclosure, it can be said that the presence or absence of a TLT in the kidney can be predicted with high accuracy when the test results obtained using biomarkers of both the sCD30 concentration in a blood sample and the eGFR match.

In addition, the sCD30 concentrations in blood samples and the eGFR from those in which any TLT is not formed in the kidney or from those in which a TLT(s) is formed in the kidney are obtained in advance. The presence or absence of a TLT may be predicted by using the analysis results of logistic regression analysis using these data.

The present inventors have found that the presence or absence of a TLT in the kidney is correlated with the creatinine concentration in a blood sample; when no TLT is formed in the kidney, the creatinine concentration is low; and when a TLT(s) is formed in the kidney, the creatinine concentration is high. Therefore, in test method 1 of the present disclosure, the creatinine concentration in a blood sample, together with the sCD30 concentration in a blood sample, may be used as a TLT biomarker to increase the accuracy of predicting the presence or absence of a TLT in the kidney.

The blood sample to be measured for the creatinine concentration may be any of whole blood, serum, or plasma, but serum is preferable.

In test method 1 of the present disclosure, the presence or absence of a TLT in the kidney may be tested based on the creatinine concentration in a blood sample by comparison with a reference value predetermined from those in which the presence or absence of a TLT has been found. Here, the “reference value” is a value serving as a reference for determining the presence or absence of a TLT, and specifically, it is the median of the creatinine concentration in blood samples obtained from those in which the presence or absence of a TLT has been found or a cut-off value calculated from the creatinine concentration.

For example, the median of the creatinine concentration in blood samples derived from those without a TLT formed in the kidney is obtained in advance, and this median is then used as a reference value. When the creatinine concentration in a blood sample of a subject is equal to or less than the reference value, it can be determined that the subject might not have any TLT formed in the kidney. In addition, for example, the median of the creatinine concentration in blood samples derived from those with a TLT formed in the kidney is obtained in advance, and this median is then used as a reference value. When the creatinine concentration in a blood sample of a subject is equal to or more than the reference value, it can be determined that the subject might have a TLT(s) formed in the kidney. In addition, for example, the creatinine concentrations in blood samples derived from those without a TLT formed in the kidney and the creatinine concentrations in blood samples derived from those with a TLT(s) formed in the kidney are obtained in advance. A cut-off value for distinguishing the presence or absence of a TLT in the kidney is determined from these creatinine concentrations, and the cut-off value is then used as a reference value. When the creatinine concentration in a blood sample of a subject is equal to or more than the cut-off value, it can be determined that the subject might have a TLT(s) formed in the kidney. Furthermore, the creatinine concentrations in blood samples from those in which any TLT is not formed in the kidney or in blood samples from those in which a TLT(s) is formed in the kidney are obtained in advance. The presence or absence of a TLT may be predicted from the creatinine concentration in a blood sample of a subject by using the analysis results of logistic regression analysis using these data.

Further, as shown in the section of Examples, 105 cases of kidney disease patients were analyzed by the present inventors. As a result, the median of the serum creatinine concentration of a kidney disease patient group in which a TLT (TLT composed of an aggregate of more than 120 lymphocytes) was formed in the kidney was 1.06 mg/dL; the median of the serum creatinine concentration of a kidney disease patient group in which no TLT was formed in the kidney or only an immature small TLT (TLT composed of an aggregate of 120 lymphocytes or less) was formed in the kidney was 0.73 mg/dL; and the cut-off value was determined to be 0.81 mg/dL at which the two groups were able to be distinguished with a sensitivity of 83% and a specificity of 61%. Therefore, in test method 1 of the present disclosure, when the serum creatinine concentration is also used as a TLT biomarker, these creatinine concentration values or the cut-off value can also be used as reference values.

### 2-2. Test Kit 1

Another embodiment of the present disclosure provides a kit (hereinafter, sometimes referred to as “test kit 1”) for testing the presence or absence of a TLT in a body, the test kit including a reagent for measuring an sCD30 concentration. Test kit 1 of the present disclosure is a test kit used for carrying out test method 1 above. The contents described in the section “2-1. Test method 1” are also incorporated in the test kit of the present disclosure.

Examples of the reagent for measuring an sCD30 concentration include an antibody that specifically binds to an sCD30. The antibody may be either a polyclonal antibody or a monoclonal antibody. In addition, the antibody may be an antibody fragment as long as it can specifically bind to an sCD30. Examples of the antibody fragment include a Fab fragment, a F (ab′)2 fragment, or a single-chain antibody (scFv). In addition, the antibody may be provided in a state of being immobilized on a solid phase carrier such as a microtiter plate or particles.

In addition, test kit 1 of the present disclosure may further include a dilution-use or reaction-use buffer solution containing a component(s) necessary for measurement, a washing liquid, a coloring reagent, or a reaction container.

Test kit 1 of the present disclosure may further contain a reagent for measuring a creatinine concentration. In a case where test kit 1 of the present disclosure includes a reagent for measuring a creatinine concentration, it is also possible to obtain eGFR of a subject, and to provide a test kit capable of predicting, with higher accuracy, the presence or absence of a TLT in a body. The reagent for measuring a creatinine concentration may include, for example, an enzyme for detecting creatinine by an enzymatic assay. Specific examples include creatininase, creatinase, sarcosine oxidase, an active oxygen detecting agent (e.g., peroxidase, 4-aminoantipyrine, N-ethyl-N-(2-hydroxy-3-sulfopropyl)-m-toluidine) etc.

Test kit 1 of the present disclosure may further contain a reagent for measuring a urinary protein concentration in addition to the reagent for measuring a creatinine concentration. When test kit 1 of the present disclosure includes a reagent for measuring a creatinine concentration and a reagent for measuring a urinary protein concentration, the eGFR of a subject can also be measured, and it is possible to provide a test kit capable of predicting, with higher accuracy, the presence or absence of a TLT in a body. Examples of the reagent for measuring a urinary protein concentration include a reagent(s) necessary for a urinary protein assay such as a Kingsbury-Clark method, a Meulemans method, a benzethonium chloride method, a Coomassie brilliant blue G250 method, or a Pyrogallol red method.

### 3. TLT Test Technology Using eGFR or Creatinine as Biomarker

### 3-1. Test Method 2

Still another embodiment of the present disclosure provides a method for testing the presence or absence of a TLT in the kidney of a subject (hereinafter, sometimes referred to as “test method 2”), the method including the step of measuring a creatinine concentration in serum collected from the subject to calculate eGFR.

In Test Method 2 of the Present Disclosure, the “Subject” is a Human or Non-human animal to be tested for the presence or absence of a TLT in the kidney. Examples of the non-human animal include non-human mammals such as primates, rats, mice, gerbils, guinea pigs, hamsters, ferrets, rabbits, cows, horses, pigs, goats, dogs, and cats. Suitable examples of the subject include a human.

Specific examples of the subject in test method 2 of the present disclosure include a kidney disease patient, a person who needs to be tested for the presence or absence of kidney disease, or a person who has received a kidney transplant. The type of kidney disease is as described in the section of “2-1. Test method 1” above.

The serum creatinine concentration can be measured by an enzymatic assay. In addition, since a creatinine assay kit is commercially available, the serum creatinine concentration can be conveniently measured using the commercially available kit.

The eGFR can be determined according to the above-described calculation formula in consideration of the serum creatinine concentration, age, and sex. In addition, in test method 2 of the present disclosure, the eGFR may use a result of a routinely performed blood test.

In test method 2 of the present disclosure, the presence or absence of a TLT in the kidney is tested using eGFR calculated from the serum creatinine concentration. As described above, the presence or absence of a TLT in the kidney is correlated with eGFR; when any TLT is not formed in the kidney, eGFR is high; and when a TLT(s) is formed in the kidney, eGFR is low. Thus, in test method 2 of the present disclosure, the presence or absence of a TLT in the kidney can be determined based on the eGFR of the subject. The eGFR is highly correlated with the presence or absence of a TLT composed of an aggregate of more than 120 lymphocytes. Therefore, test method 2 according to an embodiment of the present disclosure is performed in order to test the presence or absence of a TLT composed of an aggregate of more than 120 lymphocytes.

The method for determining the presence or absence of a TLT in the kidney on the basis of the eGFR of the subject is as described in the section of “2-1. Test method 1” above.

In test method 2 of the present disclosure, when the subject is a kidney disease patient and may be predicted to be at risk of having a TLT(s) formed in a body, it is presumed that the kidney disease has progressed. In addition, in test method 2 of the present disclosure, when the subject is a person who needs to be tested for the presence or absence of a kidney disease and may be predicted to be at risk of having a TLT(s) formed in a body, it is presumed that the kidney disease may have progressed. Further, in test method 2 of the present disclosure, when the subject is a person received a kidney transplant and may be predicted to be at risk of having a TLT(s) formed in a body, it is presumed that the function of the transplanted kidney is deteriorated.

### 3-2. Test Method 3

Still another embodiment of the present disclosure provides a method for testing the presence or absence of a TLT in the kidney of a subject (hereinafter, sometimes referred to as “test method 3”), the method including the step of measuring a creatinine concentration in a blood sample collected from the subject.

In test method 3 of the present disclosure, the test subject is as described in the section of “3-1. Test method 2”, and the blood sample to be used is as described in the section of “2-1. Test method 1”.

In test method 3 of the present disclosure, the creatinine concentration in a blood sample can be measured by an enzymatic assay. In addition, since a creatinine assay kit is commercially available, the serum creatinine concentration can be conveniently measured using the commercially available kit. In addition, in test method 3 of the present disclosure, the creatinine concentration in a blood sample may use a result of a routinely performed blood test.

In test method 3 of the present disclosure, the presence or absence of a TLT in the kidney is tested using the creatinine concentration in a blood sample. As described above, the presence or absence of a TLT in the kidney is correlated with the creatinine concentration in a blood sample; when no TLT is formed in the kidney, the creatinine concentration in a blood sample is low; and when a TLT(s) is formed in the kidney, the creatinine concentration in a blood sample is high. Thus, in test method 3 of the present disclosure, the presence or absence of a TLT in the kidney can be determined based on the creatinine concentration in a blood sample of a subject. In addition, the creatinine concentration in a blood sample is highly correlated with the presence or absence of a TLT composed of an aggregate of more than 120 lymphocytes. Therefore, test method 3 according to an embodiment of the present disclosure is performed in order to test the presence or absence of a TLT composed of an aggregate of more than 120 lymphocytes.

The method for determining the presence or absence of a TLT in the kidney on the basis of the creatinine concentration in a blood sample is as described in the section of “2-1. Test method 1” above.

In test method 3 of the present disclosure, when the subject is a kidney disease patient and may be predicted to be at risk of having a TLT(s) formed in a body, it is presumed that the kidney disease has progressed. In addition, in test method 3 of the present disclosure, when the subject is a person who needs to be tested for the presence or absence of a kidney disease and may be predicted to be at risk of having a TLT(s) formed in a body, it is presumed that the kidney disease may have progressed. Further, in test method 3 of the present disclosure, when the subject is a person received a kidney transplant and may be predicted to be at risk of having a TLT(s) formed in a body, it is presumed that the function of the transplanted kidney is deteriorated.

### 3-3. Test Kit 2

Still another embodiment of the present disclosure provides a kit (hereinafter, sometimes referred to as “test kit 2”) for testing the presence or absence of a TLT in the kidney, the test kit including a reagent for measuring a serum creatinine concentration. Test kit 2 of the present disclosure is a test kit used for carrying out test method 2 or test method 3 above. The contents described in the section “3-1. Test method 2” and “3-2. Test method 3” are also incorporated in test kit 2 of the present disclosure.

The reagent for measuring a serum creatinine concentration is as described in the section of “2-2. Test kit 1” above.

In addition, test kit 2 of the present disclosure may further include a dilution-use or reaction-use buffer solution containing a component(s) necessary for measurement, a washing liquid, a coloring reagent, or a reaction container.

### 4. Test Technology for Predicting Prognosis of Renal Function by Using sCD30 Concentration in Blood Sample as Biomarker

### 4-1. Test Method 4

Still another embodiment of the present disclosure provides a test method for predicting a prognosis of renal function in a subject (hereinafter, sometimes referred to as “test method 4”), the method including the step of measuring a creatinine concentration in a blood sample collected from the subject.

### [Subject]

In test method 4 of the present disclosure, the “subject” is a human or non-human animal for which prediction of future renal function is required. Examples of the non-human animal include non-human mammals such as primates, rats, mice, gerbils, guinea pigs, hamsters, ferrets, rabbits, cows, horses, pigs, goats, dogs, and cats. Suitable examples of the subject include a human.

The subject in test method 4 of the present disclosure may be any subject as long as prediction of future renal function is required, and specific examples thereof include a kidney disease patient or a person who has received a kidney transplant. The type of kidney disease is not particularly limited, and examples thereof include IgA nephropathy, lupus nephritis. ANCA-associated vasculitis, diabetic nephropathy, nephrosclerosis, pyelonephritis, or chronic renal failure. Among these kidney diseases. IgA nephropathy is preferable.

[Blood Sample and sCD30 Measurement]

In test method 4 of the present disclosure, the blood sample used and the method for measuring an sCD30 concentration are as described in the section of “2-1. Test method 1”.

### [to Predict Prognosis of Renal Function]

In a case where the sCD30 concentration in a blood sample is high, the risk of deterioration in renal function in the future may be high. Thus, in test method 1 of the present disclosure, it is predicted that the higher the sCD30 concentration in a blood sample, the higher the risk of deterioration in renal function in the future.

Test method 4 of the present disclosure may be used to predict the prognosis of renal function on the basis of the sCD30 concentration in a blood sample by comparing it with a reference value obtained in advance from those in which deterioration in renal function is detected during the prognosis and/or those in which deterioration in renal function is not detected during the prognosis. Here, the “reference value” is a value serving as a reference for predicting the prognosis of renal function. Specifically, it is, for instance, the median of the sCD30 concentration in blood samples as obtained from those in which deterioration in renal function is detected during the prognosis and/or those in which deterioration in renal function is not detected during the prognosis or a cut-off value obtained from the sCD30 concentrations in the blood samples.

For example, the median of the sCD30 concentration in blood samples derived from those in which deterioration of renal function is not detected during the prognosis is obtained in advance, and this median is then used as a reference value. When the sCD30 concentration in a blood sample of a subject is equal to or less than the reference value, the subject can be predicted to have a low risk of causing deterioration in renal function in the future. In addition, for example, the median of the sCD30 concentration in blood samples derived from those in which deterioration of renal function was detected during the prognosis is obtained in advance, and this median is then used as a reference value. When the sCD30 concentration in a blood sample of the subject is equal to or higher than the reference value, the subject can be predicted to have a high risk of causing deterioration in renal function in the future. In addition, for example, the median of the sCD30 concentration in blood samples derived from those in which deterioration in renal function was not detected during the prognosis and the median of the sCD30 concentration in blood samples derived from those in which deterioration in renal function was detected during the prognosis are determined in advance. A cut-off value for determining the presence or absence of deterioration in renal function during the prognosis is defined from these sCD30 concentrations, and the cut-off value is then used as a reference value. When the sCD30 concentration in a blood sample of a subject is equal to or more than the cut-off value, the subject can be predicted to have a high risk of causing deterioration in renal function in the future.

Further, the reference value (details are as described in the section of “2-1. Test method 1”) for the sCD30 concentration in a blood sample as used as an index for testing the presence or absence of a TLT described above may also be used to predict the risk of causing deterioration in renal function. For example, a cut-off value for determining the presence or absence of a TLT is used as a reference value. When the sCD30 concentration in a blood sample of a subject is equal to or more than the cut-off value, the subject can be predicted to have a high risk of causing deterioration in renal function in the future. Specifically, the section of Examples has demonstrated that from the results of tracking 105 kidney disease patients, the present inventors have confirmed that the rate of causing deterioration in renal function within 5 years is higher in the group having a serum sCD30 concentration of 36.719 ng/mL or more than in the group having a serum sCD30 concentration of less than 36.719 ng/mL. Therefore, in test method 4 of the present disclosure, the serum sCD30 concentration of 36.7 ng/mL is used as the cut-off value, and when the sCD30 concentration in a blood sample of a subject is 36.7 ng/mL or more, the subject can also be predicted to have a high risk of causing deterioration in renal function in the future.

### 4-2. Test Kit 3

Still another embodiment of the present disclosure provides a test kit (hereinafter, sometimes referred to as “test kit 3”) for predicting the prognosis of renal function in a subject, the test kit including a reagent for measuring an sCD30 concentration in a blood sample. The test kit 3 of the present disclosure is a test kit used for carrying out test method 4, and the contents described in the section “4-1. Test method 4” are also incorporated in test kit 3 of the present disclosure.

The reagent for measuring an sCD30 concentration in a blood sample is as described in the section of “2-2. Test kit 1” above.

In addition, test kit 3 of the present disclosure may further include a dilution-use or reaction-use buffer solution containing a component(s) necessary for measurement, a washing liquid, a coloring reagent, or a reaction container.

### EXAMPLES

The present disclosure is not at all limited to the description of the embodiments and the following Examples according to the present invention. Various modifications that can be easily conceived by those skilled in the art without departing from the scope of the Claims are also included in the present invention. The contents of, for instance, the literatures disclosed herein are incorporated by reference in their entirety.

### 1. Test Materials and Methods

### (1) Patients

First, 152 patients with IgA nephropathy diagnosed by renal biopsy between 2014 and 2021 at Kyoto University Hospital were retrospectively screened. Here, 105 cases were analyzed from 152 cases excluding: 5 cases in which serum or kidney biopsy tissue did not remain; 15 cases in which consent on “Nephrology Diseases Registry and Research on Disease Genes at Kyoto University Hospital” and “Nephrology and Urology Kidney Diseases Registry and Research on Disease-Related Genes at Kyoto University Medical School Hospital” (approval number. G562) was not obtained; 7 cases in which the consent was incomplete; 11 cases who received immunosuppressive therapy within 1 year before kidney biopsy; 1 case of systemic lupus erythematosus (SLE); 4 cases of rheumatoid arthritis; 1 case of purpura nephritis; 2 cases of liver cirrhosis, and 1 case in which sCD30 was abnormally high (1247 ng/mL).

### (2) Histological Assessment

Kidney biopsy specimens were fixed in formalin or Duboseq Brazil solution, embedded in paraffin, sectioned (thickness 2 μm), and stained with PAS (periodic acid-Schiff).

### (3) Immunofluorescence Staining of Kidney Biopsy Specimens

Paraffin-embedded sections (3 μm thick) were deparaffinized with xylene, rehydrated, and steam heated at 110° C. for 15 minutes. The sections were then incubated for 1 hour at room temperature in 5% serum of the same species as the secondary antibody, followed by incubation at 4° C. with the following primary antibodies: anti-CD3 antibody (catalog ab5690; Abcam, Cambridge, UK), anti-CD20 antibody (catalog 14-0202; eBioscience, San Diego, CA), anti-Ki67 antibody (catalog ab16667; Abcam), and/or anti-CD21 antibody (catalog MA5-11417: Thermo Scientific, Waltham, MA). Each primary antibody was visualized using an appropriate secondary antibody. In addition, the sections were also counterstained with DAPI. Each immuno-fluorescently stained section was analyzed under a confocal microscope (FV1000D; Olympus, Tokyo, Japan).

### (4) To Identify TLT

In this test, an aggregate of organized lymphocytes with signs of proliferation was defined as a TLT, in a manner similar to the criteria presented in Non-Patent Literature 4. Since the size of TLTs varied, an aggregate of 61 or more lymphocytes (T cells and B cells) and containing at least one Ki67 positive cell was defined as a TLT in this test. The number of TLTs was quantified and the stage was determined in a blinded manner according to the procedure described in Non-Patent Literature 5. Briefly, first, monocyte infiltration in the renal stroma was checked in a PAS-stained section. Next, the TLT was diagnosed by evaluating monocyte infiltration by performing immunofluorescence staining of 1) CD3ε and CD20, and 2) Ki67 and CD21 in two consecutive sections. After the TLT diagnosis, the TLT stages were classified according to the following criteria: i) TLT without follicular dendritic cells (FDCs) or a germinal center was defined as stage I; ii) TLT with FDCs but without a germinal center was defined as stage II; and iii) TLT with FDCs and a germinal center was defined as stage III. The FDCs were defined as cells strongly expressing CD21 within a TLT. The germinal center was defined as a microstructure containing a cluster of 16 or more Ki67 positive cells within the B cell area.

(5) to Measure Serum sCD30 Concentration

The serum sCD30 concentration in each IgA nephropathy patient was measured using a human sCD30 ELISA kit (Catalog BMS240; Invitrogen, Waltham, MA) according to the protocol provided by the manufacturer. Briefly, a sample or a standard was incubated with horseradish peroxidase-labeled anti-human sCD30 antibody for 3 hours at room temperature in wells having an anti-human sCD30 antibody adsorbed, washed, and allowed to develop color. Then, the fluorescence intensity was measured at 450 nm on a microplate reader (Sunrise Rainbow RC-R; TECAN, Mannedorf, Switzerland).

### (6) Immunosuppression Regimen

Steroid was administered to patients diagnosed as having disease activity (active lesions with proteinuria ≥0.5 g/day, cellular and fibrillar crescents, and so on) of IgA nephropathy. Immunosuppressive agents other than steroid were not used. For most patients, a dose of 0.5 to 1 g methylprednisolone was intravenously administered for three consecutive days, in 1 to 3 cycles, approximately every two months, and 0.5 mg/kg prednisolone was further orally administered every other day, and the dose was gradually reduced. In one case who was transferred to another hospital and in four cases as judged by a doctor, only oral administration of prednisolone was performed without intravenous administration of methylprednisolone.

### (7) Statistical Analysis

Statistical analysis was performed using JMP Pro software (version 15.2.0). In terms of data, categorical variables were represented by numbers (%), continuous values were represented by the median (25-75 percentiles) in tables, and box plots were used in the graphs. Each categorical variable was compared by Pearson's χ2 test, and each continuous variable was compared by Wilcoxon rank sum test. The correlation was determined using Pearson's correlation coefficient. Logistic regression was performed using age, serum sCD30 concentration, and baseline eGFR as explanatory variables to predict the presence or absence of a TLT. The ROC curve and the Youden index were used to determine the cut-off value of the serum sCD30 concentration for predicting the presence of a TLT. When a value was equal to or more than the cut-off value, the TLT was determined as positive. A P value of less than 0.05 was considered statistically significant. In addition, the COX proportional hazard model was used to analyze the relationship between the serum creatinine concentration, age, urinary protein, and serum sCD30 concentration and the incidence of an event in which eGFR decreased by 20% or more during the prognosis.

### 2. Test Results

(1) Classification into TLT Group and Non-TLT Group

Kidney biopsy PAS-stained specimens of 105 patients with IgA nephropathy were visually observed and divided into 70 cases suspected of having a TLT and 35 cases in which no TLT was observed. Immunostaining for CD3ε, CD20, CD21, and Ki67 was performed on the 70 kidney biopsy specimens suspected of having a TLT, and the presence or absence of a TLT was determined based on the expression of the TLT markers. As a result, among the 70 cases suspected of having a TLT visually. 54 cases were confirmed to have a TLT by immunostaining, and 16 cases were not found to have a TLT by immunostaining (FIG. 2). Next, the stage of TLT maturation was determined for 54 cases in which a TLT was observed by immunostaining. As a result. 46 cases were at stage I, 8 cases were at stage II, and 0 cases were at stage III (FIG. 2).

Note that among the 46 cases of stage I, 8 cases had a TLT (small TLT) formed of an aggregate of 61 or more and 120 or less lymphocytes, and 38 cases had a TLT formed of an aggregate of more than 120 lymphocytes. It was considered that the small TLT formed of an aggregate of 120 or less lymphocytes was an immature TLT and did not reflect the pathology. Therefore, in this test, the 38 cases having a TLT composed of an aggregate of more than 120 lymphocytes at stage I and 8 cases at stage II were analyzed as the “TLT group” (46 cases in total), and 35 cases in which TLT was not visually recognized, 16 cases in which any TLT was not found by immunostaining, and 8 cases having a small TLT at stage I were analyzed as the “non-TLT+small TLT group” (59 cases in total).

Table I shows the clinical features at the time of biopsy of the TLT group (46 cases) and the non-TLT+small TLT group (59 cases). Note that hematuria was scored, based on the urine sediment test result, according to the following criteria: 0: <5/HPF, 1: 5-19/HPF, 2: 10-49/HPF, 3: 49>/HPF.

TABLE 1

| | Non-TLT + small TLT group | | TLT group | | P value | |
|---|---|---|---|---|---|---|
| Number of Patients (n) | 59 | | 46 | | | |
| Sex (male/female) | 20/39 | | 22/24 | | 0.15 | Pearson |
| Age (years old) | 31 | (24-43) | 44 | (34-65) | 0.0020 | Wilcoxon |
| Body mass index (kg/m²) | 21.2 | (18.7-24.1) | 21.5 | (19.5-23.8) | 0.59 | Wilcoxon |
| Serum creatinine | 0.73 | (0.63-0.97) | 1.06 | (0.84-1.51) | <0.0001 | Wilcoxon |
| concentration (mg/dL) | | | | | | |
| eGFR (mL/min/1.73 m²) | 79.2 | (65.4-93.6) | 53.3 | (33.7-69.2) | <0.0001 | Wilcoxon |
| Urinary protein/creatinine | 0.37 | (0.13-0.70) | 0.84 | (0.31-1.74) | 0.0007 | Wilcoxon |

| ratio (g/gCr) | | | | |
|---|---|---|---|---|
| Hematuria | 1 (0-2) | 1 (0.8-2) | 0.68 | Wilcoxon |
| | Median (25^{th}-75^{th} percentile) | Median (25^{th}-75^{th} percentile) | | |
| Diabetes (%) | 5 | 13 | 0.15 | Pearson |
| Hypertension (%) | 15 | 50 | 0.0001 | Pearson |

(2) Comparison Between Presence or Absence of TLT and Serum sCD30 Concentration in IgA Nephropathy

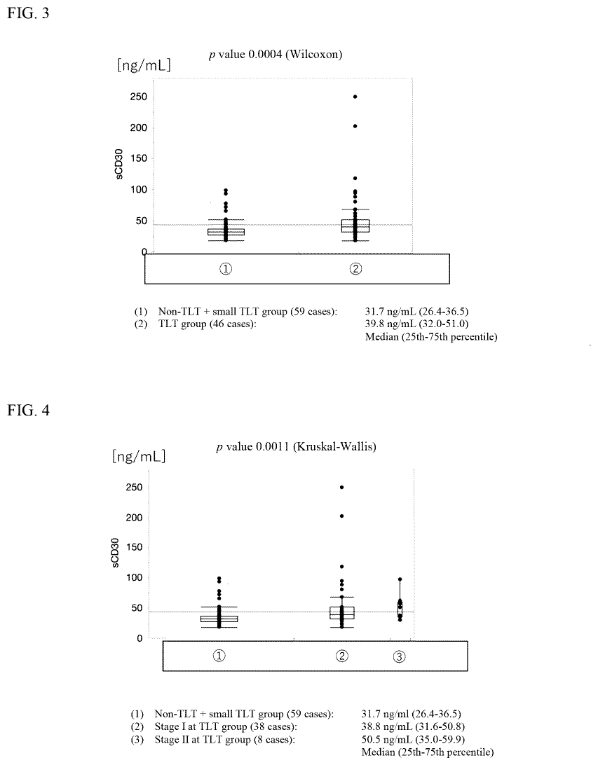

When the serum sCD30 concentration was compared between the TLT group and the non-TLT+small TLT group, the serum sCD30 concentration was significantly higher in the TLT group than in the non-TLT+small TLT group (FIG. 3). In addition, the TLT group was divided into stage I (38 cases) and stage II (8 cases), and the serum sCD30 concentrations were compared. As a result, the serum sCD30 concentration was higher in stage II than in stage I (FIG. 4). That is, it has been found that the serum sCD30 concentration may serve as a biomarker for determining the presence or absence of a TLT in IgA nephropathy.

(3) Correlations Between Serum sCD30 Concentration and Serum Creatinine Concentration, Urinary Protein/Creatinine Ratio, and Age in IgA Nephropathy

The correlations were checked between the serum sCD30 concentration and the serum creatinine (sCre) concentration, the urine protein/creatinine ratio (UPCR), and the age in 105 cases of IgA nephropathy. As a result, it was found that in IgA nephropathy, the serum sCD30 concentration had a weak positive correlation with the serum sCre concentration, UPCR, or age (FIG. 5).

(4) Prediction of Presence or Absence of TLT by Logistic Regression Using Serum sCD30 Concentration, eGFR and/or Serum Creatinine Concentration

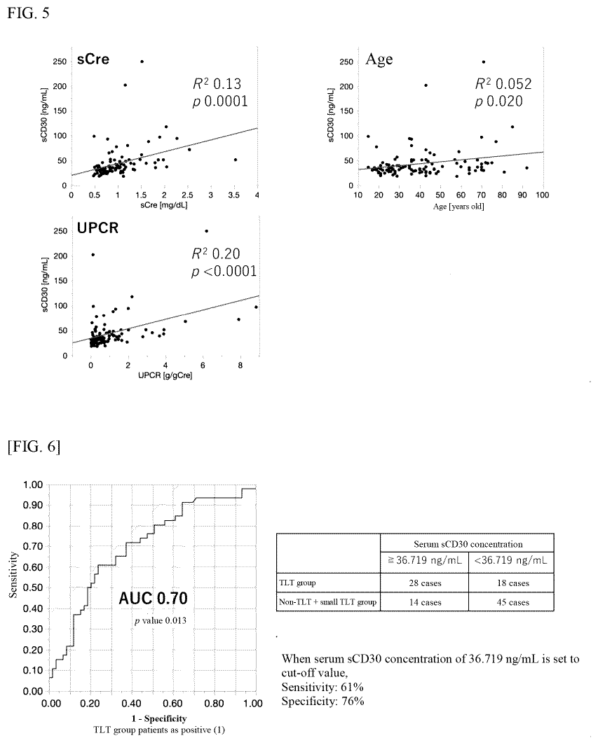

Univariate analysis was conducted by logistic regression while using the serum sCD30 concentration as an explanatory variable, in which analysis patients in the TLT group were set as positive (1) and patients in the non-TLT+small TLT group were set as negative (0) in 105 cases of IgA nephropathy. As a result, the AUC (Area Under Curve) was 0.70, and the serum sCD30 concentration at which the “Sensitivity−(1−Specificity)” was maximum was 36.719 ng/mL (FIG. 6). When that serum sCD30 concentration was used as a cut-off value, the sensitivity was 61% and the specificity was 76% (FIG. 6).

In addition, univariate analysis was conducted by logistic regression while using the serum creatinine concentration as an explanatory variable, in which analysis patients in the TLT group were set as positive (1) and patients in the non-TLT+small TLT group were set as negative (0) in 105 cases of IgA nephropathy. As a result, the AUC was 0.76, the serum creatinine concentration at which the “Sensitivity−(1−Specificity)” was maximum was 0.81 mg/dL When that serum creatinine concentration was used as a cut-off value, the sensitivity was 61%, and the specificity was 83% (the figure is omitted).

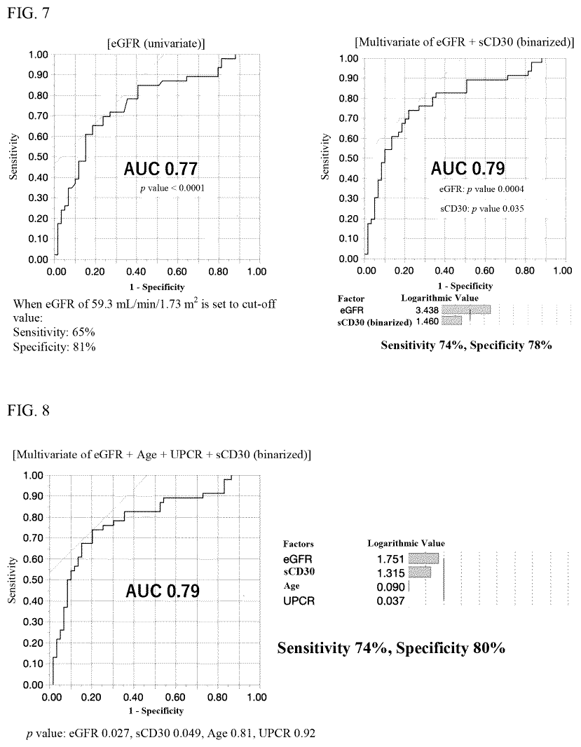

Univariate analysis was conducted by logistic regression while using the eGFR as an explanatory variable, in which analysis patients in the TLT group were set as positive (1) and patients in the non-TLT+small TLT group were set as negative (0) in 105 cases of IgA nephropathy. As a result, the AUC was 0.77, and the eGFR at which the “Sensitivity−(1−Specificity)” was maximum was 59.3 mL/min/1.73 m² (left graph in FIG. 7). When that eGFR was used as a cut-off value, the sensitivity was 65% and the specificity was 81% (left graph in FIG. 7).

In addition, the presence or absence of a TLT was predicted from multivariate analysis by logistic regression using eGFR and sCD30 (the serum sCD30 concentration) after binarized (<36.719 ng/mL: 0, ≥36.719 ng/mL: 1) for 105 cases of IgA nephropathy. As a result, this model had an AUC of 0.79, a sensitivity of 74%, and a specificity of 78% (right graph in FIG. 7).

Further, the presence or absence of a TLT was predicted from multivariate analysis by logistic regression using eGFR, age, UPCR, and the serum sCD30 concentration after binarized (<36.719 ng/mL: 0, ≥36.719 ng/mL: 1) for 105 cases of IgA nephropathy. As a result, this model had an AUC of 0.79, a sensitivity of 74%, and a specificity of 80% (FIG. 8). No significant difference was given by age and UPCR, which were considered to contribute a little to the prediction of the presence of a TLT.

Table 2 collectively provides the results of analyzing the presence or absence of a TLT by logistic regression using the baseline eGFR, age, and/or serum sCD30 concentration as explanatory variables. The results of this logistic regression analysis have also demonstrated that the serum sCD30 concentration can independently be a predictor of a TLT.

TABLE 2

| | Univariate logistic | | Multivariate logistic | |
|---|---|---|---|---|
| | regression | | regression | |
| | Odds ratio | | Odds ratio | |
| | (95% CI) | P value | (95% CI) | P value |
| Serum | 0.960 | <0.0001 | 0.970 | 0.024 |
| creatinine | (0.942-0.979) | | (0.945-0.996) | |
| concentration | | | | |
| (+1 mg/dL) | | | | |
| Age (+1 | 1.036 | 0.0026 | 1.004 | 0.80 |
| years old) | (1.013-1.061) | | (0.972-1.038) | |
| Serum sCD30 | 5.00 | 0.0002 | 2.84 | 0.034 |
| concentration | (2.15-11.6) | | (1.09-7.44) | |
| (≥36.719 ng/mL | | | | |
| or <36.719 ng/mL) | | | | |

(5) Correlation Between Prognosis of IgA Nephropathy and Serum sCD30 Concentration

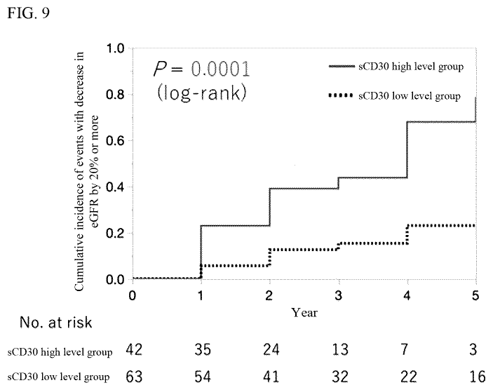

When 105 cases of IgA nephropathy were followed for 5 years, the event, in which eGFR decreased by 20% or more as compared with the value at the time of kidney biopsy, occurred in 27 cases. For the 105 cases of IgA nephropathy, a hazard ratio for incidence of an event in which eGFR decreased by 20% or more was determined by a COX proportional hazard model using the serum creatinine concentration, age, urine protein/creatinine ratio, and serum sCD30 concentration (binarized: <36.719 ng/mL: 0; ≥36.719 ng/mL: 1). FIG. 9 is a graph showing the Kaplan-Meier method results of tracking the presence or absence of incidence of an event in which eGFR decreases by 20% or more over 5 years while dividing 105 cases of IgA nephropathy into the sCD30 high level group (serum sCD30 concentration is 36.719 ng/mL or more) and the sCD30 low level group (serum sCD30 concentration is less than 36.719 ng/mL). The results have revealed that the sCD30 high level group had higher incidence of an event in which eGFR decreased by 20% or more, and the serum sCD30 concentration can thus be a predictive marker for the prognosis of renal function.

TABLE 3

| | Univariate COX | | Multivariate COX | |
|---|---|---|---|---|
| | proportional | | proportional | |
| | hazard model | | hazard model | |
| | Hazard ratio | P | Hazard | P |
| | (95% CI) | (Wald) | (95% CI) | (Wald) |
| Baseline eGFR | 1.77 | 0.023 | 0.753 | 0.45 |
| (+1 mL/min/ | (1.008-2.757) | | (0.330-1.48) | |
| 1.73 m²) | | | | |
| Age | 1.024 | 0.018 | 1.015 | 0.20 |
| (+1 years old) | (1.003-1.044) | | (0.992-1.038) | |
| Urinary protein/ | 1.434 | <0.0001 | 1.31 | 0.012 |
| creatinine ratio | (1.202-1.627) | | (1.044-1.606) | |
| (+1 g/gCr) | | | | |
| Serum sCD30 | 3.99 | 0.0008 | 3.09 | 0.015 |
| concentration | (1.77-8.99) | | (1.24-7.69) | |
| (≥36.719 ng/mL | | | | |
| or <36.719 ng/mL) | | | | |

1. A test method for testing a presence or absence of a tertiary lymphoid tissue in a subject, comprising the step of measuring a soluble CD30 concentration in a blood sample collected from the subject.

2. The test method according to claim 1, wherein the subject is a kidney disease patient, a person who needs to be tested for a presence or absence of kidney disease, or a person who has received a kidney transplant, and the presence or absence of a tertiary lymphoid tissue in a kidney is tested.

3. The test method according to claim 1, wherein the blood sample is serum.

4. The test method according to claim 1, further comprising a step of measuring a creatinine concentration in serum collected from the subject to calculate an estimated glomerular filtration rate of the subject.

5. A test kit for testing a presence or absence of a tertiary lymphoid tissue in a body, comprising a reagent for measuring a soluble CD30 concentration.

6. The kit according to claim 5, further comprising a reagent for measuring a creatinine concentration.

7. A method for testing a presence or absence of a tertiary lymphoid tissue in a kidney of a subject, comprising the step of measuring a creatinine concentration in serum collected from the subject to calculate an estimated glomerular filtration rate.

8. A method for testing a presence or absence of a tertiary lymphoid tissue in a kidney of a subject, comprising the step of measuring a creatinine concentration in serum collected from the subject.

9. A test kit for testing a presence or absence of a tertiary lymphoid tissue in a kidney, comprising a reagent for measuring a creatinine concentration.

10. A test method for predicting a prognosis of renal function in a subject, comprising the step of measuring a soluble CD30 concentration in a blood sample collected from the subject.

11. The test method according to claim 10, wherein the subject is a kidney disease patient or a person who has received a kidney transplant.

12. A test kit for predicting a prognosis of renal function in a subject, comprising a reagent for measuring a soluble CD30 concentration.

13. The test method according to claim 2, wherein the blood sample is serum.

14. The test method according to claim 2, further comprising a step of measuring a creatinine concentration in serum collected from the subject to calculate an estimated glomerular filtration rate of the subject.
